(19) United States
(12) Patent Application Publication
(10) Pub. No.: US 2011/0230863 A1
(43) Pub. Date: Sep. 22, 2011

(54) POST-SURGICAL DRAIN FACILITATING BELT AND METHOD OF USE

(76) Inventor: NEDDA JOY LENTINI, Monterey, CA (US)

(21) Appl. No.: 13/089,292

(22) Filed: Apr. 18, 2011

Related U.S. Application Data
(63) Continuation-in-part of application No. 11/805,745, filed on May 24, 2007, now Pat. No. 7,942,856.
(60) Provisional application No. 60/808,004, filed on May 25, 2006.

(51) Int. Cl. A61M 27/00

(52) U.S. Cl. 604/541

(57) ABSTRACT

A garment and a belt for securing and storing bodily fluid drainage reservoirs and other fluid reservoirs are provided, and a method of use thereof. The belt has at least one small aperture for receiving drainage tubing and at least one pocket cloth partly fixed to the belt and partly detachably coupled to the belt which allows for the easy storage and removal of the drainage reservoir or other fluid reservoir. The pocket cloth allows for the continuing support of the drainage reservoir as the drainage reservoir becomes heavier, e.g., about 1 kilogram, as body fluid drains into the drainage reservoir. The secure storage of the drainage reservoir enables the wearer to move without reduced worry of the drainage reservoir coming loose, losing fluid, or being exposed to a non-sterile environment external to the pocket cloth. The belt may be configured for wear by male and female children and adults.

The present U.S. Nonprovisional patent application is a continuation in part from both U.S. Nonprovisional patent application Ser. No. 11/805,745 titled “JACKSON-PRATT™ post surgical drain facilitator gown” filed on May 24, 2007 and U.S. Provisional Patent Application No. 60/808,004 filed May 25, 2006. The present U.S. Patent Application claims benefit of the priority date of U.S. Nonprovisional patent application Ser. No. 11/805,745 and U.S. Provisional Patent Application No. 60/808,004.

### BACKGROUND OF THE INVENTION

1. Field of the Invention

This invention relates generally to the field of post-surgical garments, and more particularly to a user wearable belt to be worn by a wearer who has undergone a medical procedure such as surgery, for example a mastectomy or axillary node dissection.

2. Description of the Related Art

A JACKSON-PRATT™ surgical drain is a device that suctions and collects fluid from a post-surgical patient's surgery site. The JACKSON-PRATT™ drainage tubing is commonly inserted at surgery sites near the patient's underarm area, near the breast or near the patient's thigh area. The drain allows for the collection of fluid until the patient's body is able to reabsorb the fluid on its own. The drain also allows for the measurement of the amount of fluid draining from the patient's body, an important indicator as to whether the patient is hemorrhaging or experiencing a clot. For these reasons, health care professionals closely monitor the amount of fluid draining into the JACKSON-PRATT™ drain bulb.

The complete drainage reservoir assembly comprises drainage tubing which is inserted into the patient's surgery site at one end and is attached to a JACKSON-PRATT™ drainage bulb at the other end, allowing fluid to travel from the patient's body into the drainage bulb. The JACKSON-PRATT™ drainage assembly is often secured to a standard hospital gown by feeding the tubing through an opening of the gown and pinning the drainage reservoir bulb to the exterior portion of the gown. As the JACKSON-PRATT™ drainage bulb fills with fluid, it becomes heavier and harder to secure to a standard hospital gown. Often the weight of the drain drags the hospital gown downward, causing the ties securing the patient's gown to loosen or come undone. Also, the pin may detach from the gown causing the drain to come loose. This is extremely dangerous for a post-surgical patient for at least two reasons.

First, if the drainage reservoir assembly separates from the hospital gown the drainage reservoir bulb may drop on the ground and become exposed to a non-sterile environment. Exposure of the drainage reservoir assembly to a non-sterile environment may lead to infection in the patient. This is especially true for a patient who has just experienced surgery and has an open surgical site.

Second, fluid may spill from the drainage reservoir bulb so that the patient's healthcare provider is unable to monitor how much fluid is collecting in the drainage reservoir bulb. The amount of fluid collecting in the drainage reservoir bulb is an important indicator as to how much fluid the patient is absorbing. Often high fluid levels indicate that a patient may be hemorrhaging, whereas low fluid levels indicate that a patient may be experiencing a clot. If the drain assembly becomes loose and fluid spills from the drain bulb the patient's doctor or nurse may be unable to determine whether the patient is hemorrhaging or clotting.

Each and every other patent and patent application mentioned in this disclosure, to include U.S. Pat. Nos. 5,429,593; 5,643,233; 5,980,499; 6,032,289; 6,574,800; 7,010,812; 7,073,204; and 6,524,288, are incorporated in their entirety and for all purposes in the present patent application and this disclosure. Other U.S. patents have suggested approaches for supporting fluid drainage devices. These include U.S. Pat. No. 5,643,233, U.S. Pat. No. 5,429,593, U.S. Pat. No. 5,980,499, U.S. Pat. No. 6,524,288, and U.S. Pat. No. 5,429,593. While these devices do serve to support a fluid drainage assembly, the devices are meant for wear underneath clothing. The wearer must partially or completely disrobe to access the fluid drainage devices. In a hospital setting, it is important to facilitate access to the fluid drainage devices so that health care providers may rapidly and frequently assess a patient's fluid absorption. If the fluid drainage assembly is difficult to access, then there may be a risk that the fluid drainage assembly will be examined less frequently and changes in fluid absorption may go unnoticed.

Other U.S. patents, such as U.S. Pat. No. 7,010,812, suggest approaches for supporting fluid drainage devices on the outside of the garment. However, these devices only support the fluid drainage device near the wearer's breast region. It is important that the fluid drainage reservoir not be too elevated in comparison to the wearer's surgical site and drainage tubing. The elevated placement of the fluid drainage reservoir may inhibit the flow of fluid from the surgical site into the fluid drainage bulb. This decreases the efficacy of the medical drainage device and increases the risk of infection in the patient.

Other U.S. patents, such as U.S. Pat. No. 6,574,800, suggest approaches for supporting fluid drainage devices on the outside of the garment with lower fluid drainage device support. However, these devices are configured for home recovery and do not possess a pocket cloth which is partly fixed to the garment fabric and partly detachably coupled. Without a pocket that is partly detachably coupled to the garment fabric, the device does not provide the same ease of access to the medical drainage device that is important in a hospital setting. A partly detachably coupled pocket cloth facilitates ease of access to the drainage reservoir bulb so that the drainage reservoir bulb is more easily monitored and changed by the wearer or a healthcare provider.

Thus, it is found that the various techniques and configurations commonly employed for supporting a fluid drainage device fail to meet the needs of a patient in a hospital setting. There is therefore a long felt need to provide methods and systems that support the use of bodily fluid reservoirs. The present invention meets a long felt need for a garment that facilitates access to the fluid drainage device, and secures and supports the fluid drainage device which reduces, minimizes, or eliminates the risk of the fluid drainage device coming into contact with a non-sterile environment or spilling fluid.

### SUMMARY OF THE INVENTION

Towards this object and other objects that will be made obvious in light of this disclosure, the method of the present invention provides a garment and a belt that supports the use of a bodily fluid reservoir. The present invention provides an improved drainage reservoir support garment or belt which secures and supports JACKSON-PRATT™ drainage reservoirs and other fluid reservoir assemblies without the aid of pins or tape. The present invention is directed to a garment and a belt including a pocket cloth or set of pocket cloths attached to a fabric sheet. The pocket cloths may be attached to the fabric at locations close to or covering apertures in the fabric of the gown or belt. The apertures may be large enough to receive drainage tubing approximately 1 cm thick and a drainage reservoir bulb approximately 10 cm in diameter and allow drainage tubing to be fed from the wearer's surgical area through the opening of the garment or belt and into the interior of the pocket cloth securing the JACKSON-PRATT™ drainage reservoir or other fluid reservoir. The pocket cloths may be secured to the belt or body of the garment or belt in a manner which allows a portion of the pocket cloth to be partly fixed to the fabric of the garment or belt, such as but not limited to partly sewing or gluing the pocket cloth to the fabric of the garment or belt, and a portion of the pocket cloth to be detachably coupled to the fabric with at least one releasable fastener, such as but not limited to snaps, buttons, ties, hook and loop, and zippers. Thus, a portion of the pocket cloth may be in certain embodiments decoupled from the body, allowing for easier access to the JACKSON-PRATT™ drainage bulb when restrained by the belt or gown.

In certain preferred alternate embodiments, the pocket cloths are sufficient in size to accommodate a drainage reservoir bulb approximately 10 cm in diameter. The present invention facilitates access to the drainage reservoir assembly by allowing access to the assembly from the exterior of the garment or belt in certain still alternate preferred embodiments. Because the pocket cloths are partly detachably coupled, a portion of the pocket cloth may rapidly and effortlessly be opened and the drainage reservoir assembly quickly examined or changed. In a hospital setting, the drainage reservoir assembly may need to be examined and changed frequently by the wearer or healthcare provider. Facilitating ease of access to the drainage reservoir assembly ensures that the drainage reservoir assemblies are frequently monitored which in turn minimizes risk of infection to the wearer.

Pocket cloths may be attached near the garment or belt wearer's (a.) chest region; (b.) thigh region; (c.) waist region; or (d.) torso. Surgical incisions may exist at numerous locations on the wearer's body. By locating the pocket cloth lower on a waist belt or the garment near the wearer's thigh region or waist region, fluid from a higher torso or upper body incision site on the wearer's body does not need to travel upwards against gravity to an elevated location on the garment or belt. This facilitates drainage and minimizes the risk of infection.

By securing and supporting JACKSON-PRATT™ drainage reservoir or other fluid reservoir with the pocket cloth rather than with pins or tape, the present invention reduces, minimizes, or eliminates the risk of the drainage assembly from coming into contact with a non-sterile environment. The present invention may also reduce, minimize, or eliminate the risk of fluid spilling from the drainage bulb.

Certain yet alternate preferred embodiments of the present invention are directed to a method of providing a garment or a belt having a pocket configured for securing a fluid reservoir, comprising a tubing aperture, the tubing aperture enabling placement and removal of the fluid reservoir through the interior side of the garment or belt; coupling a fluid reservoir to a wearer's body; and placing the fluid reservoir through the tubing aperture, whereby a tubing extends through the tubing aperture.

### BRIEF DESCRIPTION OF THE DRAWINGS

FIG. 1 is a perspective representation of the front view of the garment of the present invention showing a set of four pocket cloths for storing a drainage reservoir;

FIG. 2 is a perspective representation showing the back of the garment;

FIG. 3 is a perspective representation of the pocket cloth for storing a drainage reservoir, showing a portion of the pocket cloth sewn to the fabric of the garment and a portion of the pocket cloth detachably coupled to the fabric of the garment with releasable fasteners (e.g. snaps, buttons, ties, hook and loop, hook and eye, buckles, and zippers);

FIG. 4 is a perspective representation of the pocket cloth for storing a drainage reservoir showing the open pocket cloth and showing an aperture in the fabric of the garment which the pocket cloth had been covering;

FIG. 5 is a perspective representation showing the pocket cloth and showing an aperture in the fabric of the garment which is positioned above the pocket.

FIG. 6 is a front perspective view of an invented belt for carrying and securing the fluid drainage reservoir of FIGS. 3 and 5;

FIG. 7 is a back perspective view of the invented belt of FIG. 6; and

FIG. 8 is a front view of the belt of FIGS. 6 and 7.

### DETAILED DESCRIPTION OF THE PREFERRED EMBODIMENTS

The accompanying drawings are referenced in conjunction with the detailed description so that the present invention may be more readily understood. The present invention provides a user wearable garment 10 capable of securing and storing drainage reservoirs for a post operative wearer.

The garment 10 comprises a fabric sheet with a front side 11 and a back side 16, a head opening and two arm openings. The body of the garment is large enough to loosely fit around a torso region of a wearer's body and extend to about the knee region of the wearer's body. The garment 10 is detachably coupled around the wearer's body so that the wearer may easily put on and remove the garment.

The pocket cloths 12 are attached to the fabric at locations close to or covering apertures 15 in the fabric of the gown. The apertures 15 are within a range of 0 cm to 15 cm and are large enough to receive drainage tubing approximately 1 cm thick and a drainage reservoir bulb approximately 10 cm in diameter and allow drainage tubing to be fed from the wearer's surgical area through the opening of the garment and into the interior of the pocket cloth 12 securing a JACKSON-PRATT™ drainage reservoir or other suitable fluid reservoir known in the art. The pocket cloths 12 are sufficient in size to accommodate a drainage reservoir bulb approximately 10 cm in diameter and the length and width of the pocket cloths 12 is within a range of 0 cm to 20 cm.

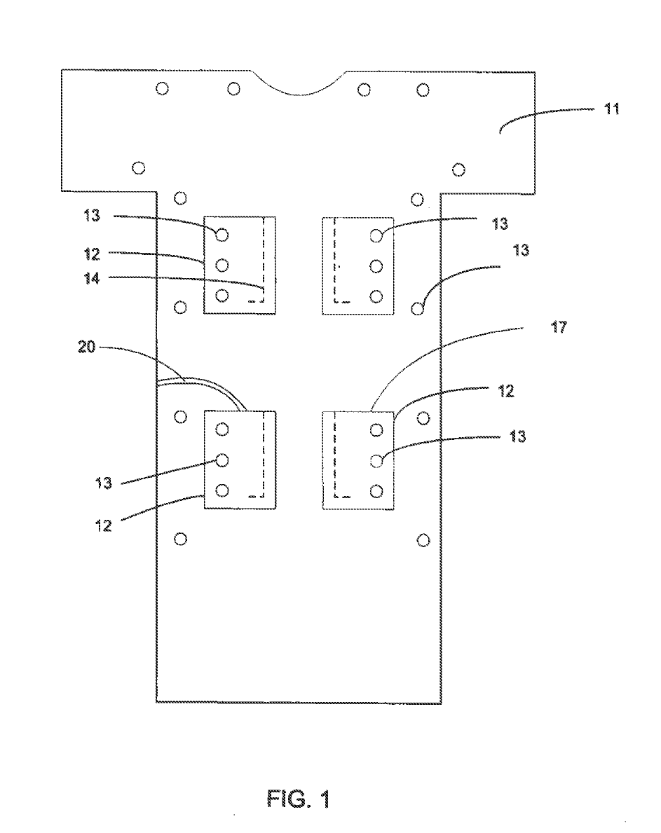

FIG. 1 shows a user wearable garment made of a fabric sheet. This embodiment shows the front side 11 of the garment. The garment has a head opening, two arm openings, and short sleeves. This embodiment uses releasable fasteners 13 at the top and sides to detachably couple the front side 11 of the garment to the back side 16 of the garment. Although these releasable fasteners 13 are shown as snaps, the releasable fasteners 13 utilized in the present invention could be any suitable releasable closing structures such as but not limited to snaps, buttons, ties, hook and loops, hook and eyes, buckles, and zippers. In this embodiment drainage tubing 20 runs in between a side aperture formed between two releasable fasteners detachably connecting the front side 11 of the garment and the back side 16 of the garment and into a pocket cloth 12.

In this embodiment, two pocket cloths 12 are located proximate to, e.g. within 20 cm of, the wearer's chest region and two pocket cloths 12 are located proximate to, e.g. within 20 cm of, the wearer's thigh region. Each pocket cloth 12 comprises a piece of material not more than 20 cm long and 20 cm wide sewn to the garment fabric along one side of the pocket cloth 12 and half way across the bottom of the pocket cloth 12. The pocket cloth 12 could be fixed to the fabric by means other than sewing. For example, the pocket cloth 12 could be partly fixed to the fabric using glue. Opposite the side of the pocket cloth 12 partly fixed to the fabric, the pocket cloth is detachably coupled to the fabric of the garment utilizing releasable fasteners 13. In the present embodiment, the releasable fasteners 13 are snaps. In this embodiment, drainage tubing is fed through an aperture between releasable fasteners 13 at the side of the garment and into the lower left pocket formed between the fabric and the pocket cloth 12. A top aperture 17 exists between the garment fabric and the pocket cloth 12.

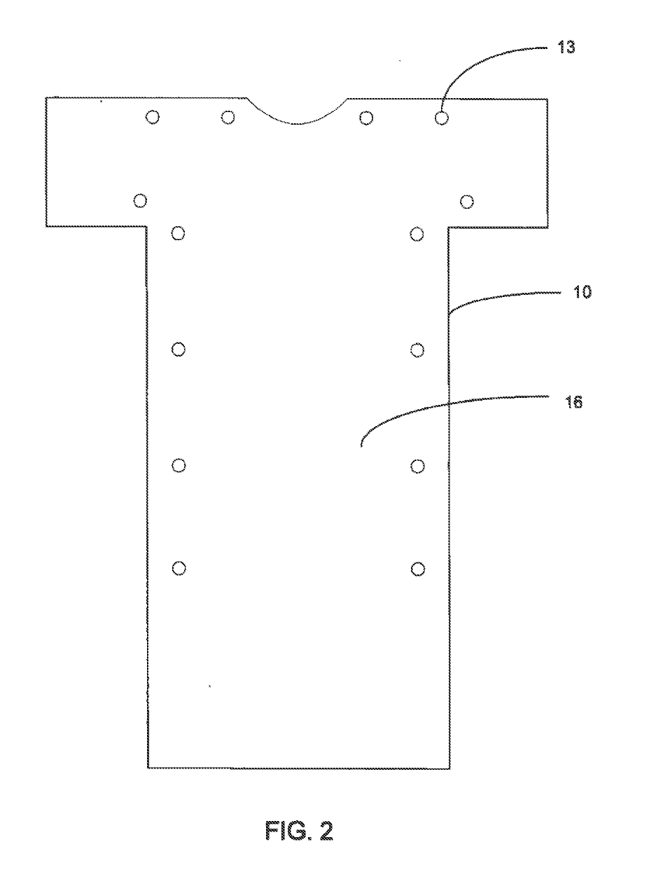

FIG. 2 shows the back side 16 of the garment. Releasable fasteners 13 are utilized to secure the front side 11 of the garment to the back side 16. Although these releasable fasteners 13 are shown as snaps, the releasable fasteners 13 utilized in the present invention could be any suitable releasable closing structures such as but not limited to the group consisting of snaps, buttons, ties, hook and loop, hook and eye, buckles, and zippers. The back side may be comprised of two separate panels including a left panel and a right panel detachably coupled together utilizing releasable fasteners, but this is not the preferred embodiment because the ties often become loose as the weight of the drainage reservoirs in the front increases.

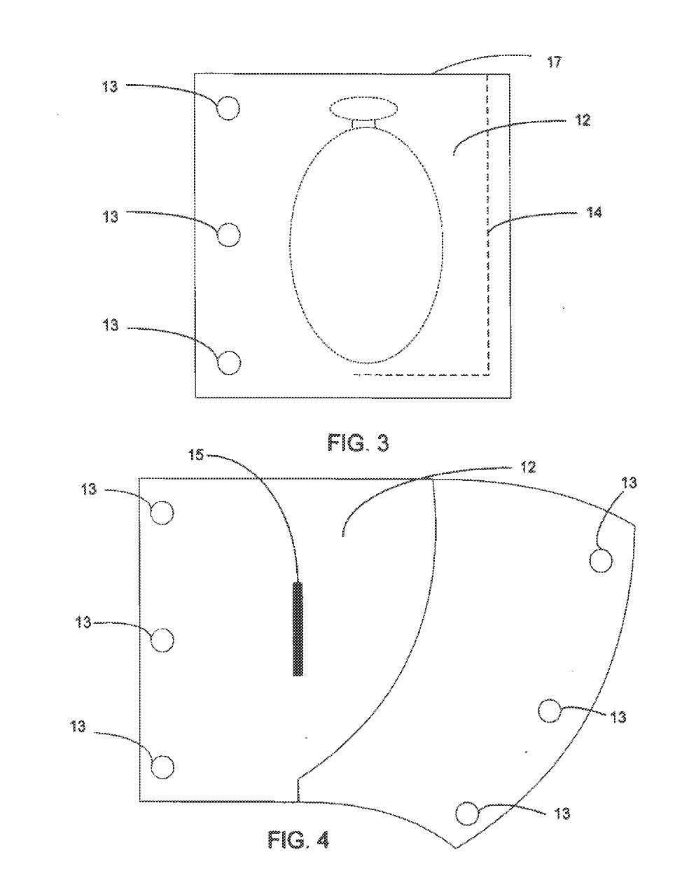

FIG. 3 is a closer perspective view of the pocket cloth 12 in the closed position. The pocket cloth 12 is partly fixed to the garment by a sewing 14 positioned along one side of the pocket cloth 12 and half way across the bottom of the pocket cloth 12. The opposite side of the pocket cloth 12 is detachably coupled to the garment fabric utilizing releasable fasteners 13. This embodiment utilizes snaps, but any suitable releasable closing structures could be used such as but not limited the group consisting of snaps, buttons, ties, hook and loop, hook and eye, buckles, and zippers. A top aperture 17 exists between the garment fabric and the pocket cloth 12.

FIG. 4 shows a closer perspective view of the pocket cloth 12 in the open position. The pocket cloth 12 is partly fixed 14 to the fabric by sewing along one side of the pocket cloth 12 and half way across the bottom of the pocket cloth. Releasable fasteners 13 are shown along one side of the pocket cloth 12 and along the corresponding side of the garment fabric at an attachment site. An aperture 15 in the garment fabric allows drainage tubing to travel from the wearer's surgical site through the aperture 15 and into the pocket formed between the fabric of the garment and the pocket cloth 12 where the drainage reservoir bulb is stored. This embodiment shows the aperture 15 in the fabric of the garment underneath the pocket cloth, but the aperture 15 could be located proximate to the pocket cloth 12 such as but not limited to directly above the pocket cloth 12. The aperture 15 in the fabric is between 0.5 cm and 12.0 cm in length.

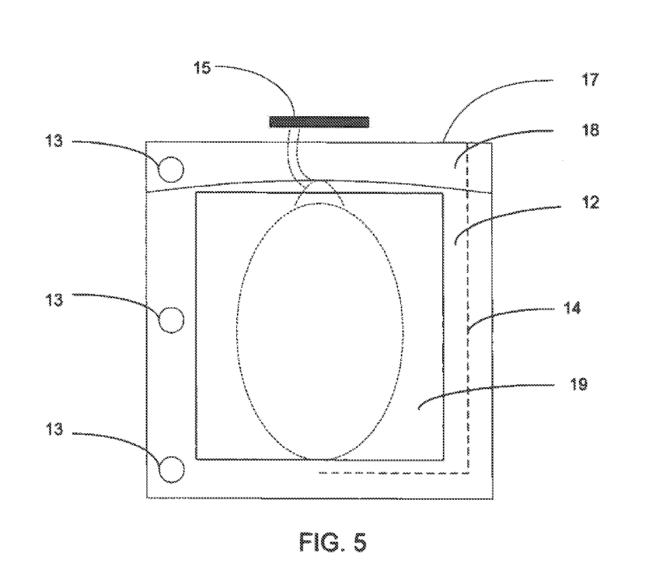

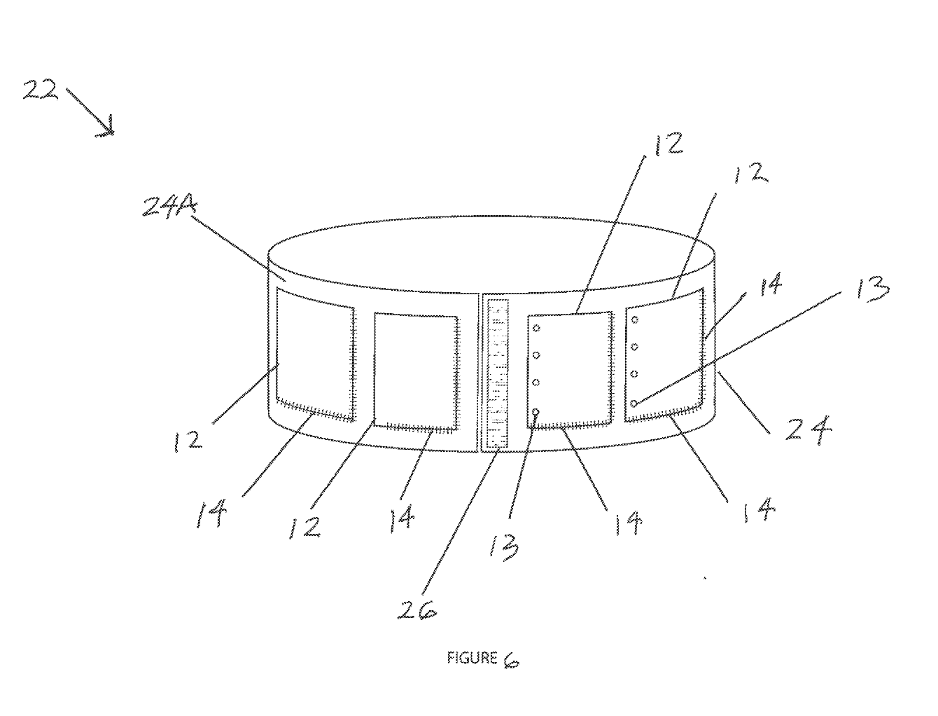

FIG. 5 shows a closer perspective view of the pocket cloth 12 in the closed position. In this embodiment the pocket cloth 12 is partly fixed 14 to the garment fabric by sewing along one side of the pocket cloth 12 and half way across the bottom of the pocket cloth. The opposite side of the pocket cloth 12 is detachably coupled to the garment fabric utilizing releasable fasteners 13. The aperture 15 in the garment fabric is located directly above the pocket cloth 12. The drainage tubing for the drainage reservoir assembly extends from the wearer's body though the aperture and down into the pocket formed between the garment fabric and the pocket cloth 12 to the drainage reservoir bulb. A top aperture 17 exists between the garment fabric and the pocket cloth 12. An insertable rigid shield 19 surrounds the drainage reservoir bulb. An elastic material 18 forms the top of the pocket cloth 12 so that the top portion of the pocket cloth 12 may retract more than the bottom portion of the pocket cloth 12.

The garment may be constructed of a breathable material utilizing, but not limited to, cotton or fabric blend material, organic fibers and/or synthetic fabric or fibers. In one preferred embodiment, the garment may be constructed of material comprising 55% cotton and 45% polyester. The fabric must provide the necessary support to hold a full drainage reservoir bulb.

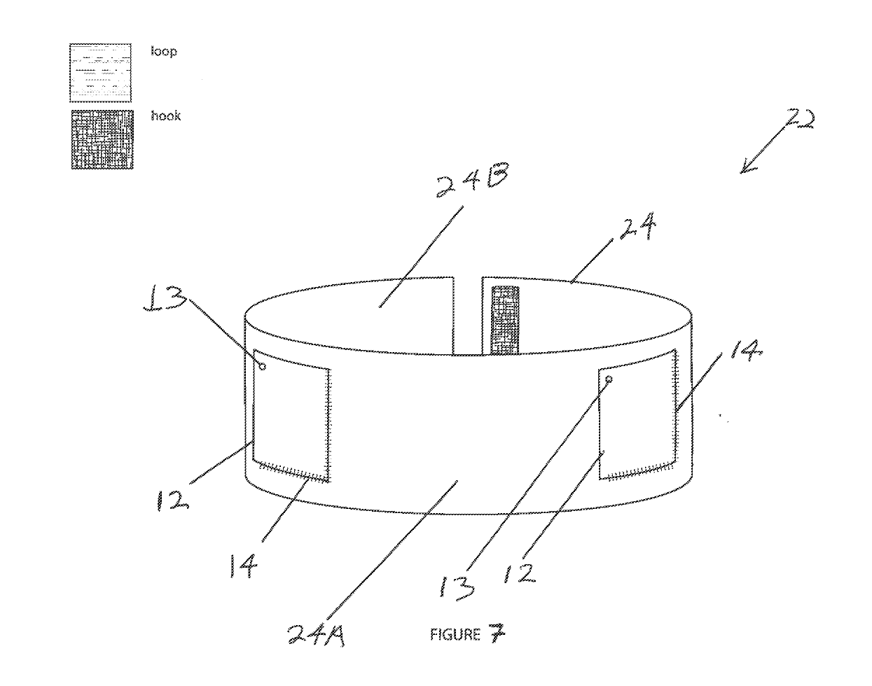

Referring now generally to the Figures and particularly to FIG. 6, FIG. 6 is a front perspective view of an invented belt 22 configured in accordance with additional aspects of the method of the present invention. Four pocket cloths 12 are shown partially sown along distinguishable thread lines 14 onto a belt material 24. Releasable fasteners 13 are also employed to releasably further attach two of the pocket cloths 12 to the belt material 24. A first hook and loop belt fastener strip 26 is further attached to a front side 24A of the belt material 24 to enable detachable securing of the belt 22 around a wearer's body.

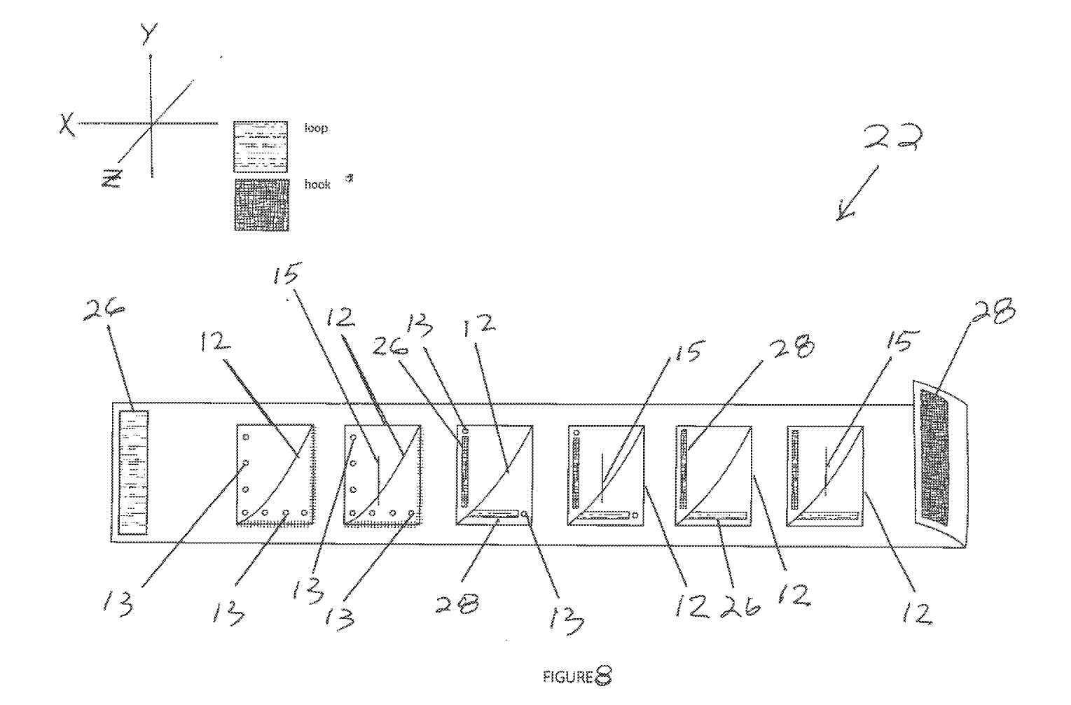

Referring now generally to the Figures and particularly to FIG. 7, FIG. 7 is a back perspective view of the invented belt 22. Two pocket cloths 12 are attached to the belt material 24 by additional distinguishable lengths of thread lines 14. Each pocket flap 12 is releasably further attached to the belt material 24 by a single dedicated releasable fastener 13.

Referring now generally to the Figures and particularly to FIG. 8, FIG. 8 is a front view of the belt 22 of FIGS. 6 and 7, wherein the six individual pocket cloths 12 of FIGS. 6 and 7 are presented. A second hook and loop belt fastener strip 28 is shown to be attached to a back side 24B of the belt material 24. The first hook and loop belt fastener strip 26 and the second hook and loop belt fastener strip 28 are selected and matched to enable detachable securing of the belt 22 around a wearer's body. A plurality of separate and individual apertures of the belt material 24 enable the draining tubing 20 to extend through the belt material 24. In addition, additional pairs of hook and loop strips 26 & 28 are employed to detachably further secure one or more pocket cloths 12 to the belt material 22.

The pocket cloths 12 and belt material 24 are preferably water resistant or waterproof, an more preferably permit moisture to travel from the back side 24B of the belt material 24 to the front side 24A of the belt material 24, and further inhibit or prohibit transfer of moisture from the front side 24A of the belt material 24 and towards the back side 24B of the belt material 24. The belt material 24 may be or comprise nylon, GORE-TEX™, cotton, natural fibers of fabrics, and/or synthetic fibers or fabrics.

The belt 22 preferably weighs less than two pounds when empty of fluid and fluid reservoirs, and most preferably less than one pound. The belt 22 preferably extends in along the Y-axis in the range of from one inch to six inches. The belt material 24 preferably has a thickness along the Z-axis of less than 0.25 inch and most preferably of less than 0.125 inch. In addition, the belt material 24 further is dimensioned along the X-axis to enable a snug but comfortable fit around the wearer's body when the first hook and loop belt fastener strip 26 coupled and engaged with the second loop belt fastener strip 28 while the belt material simultaneously encompasses a portion of the wearer's body, e.g., the wearer's waist.

While the invention has been described with reference to preferred and example embodiments, other variations and modifications within the scope of the invention will become apparent to those of skill in the art. Such variations are included within the spirit and scope of this invention as defined by the following claims.

I claim:

1. A belt, comprising:
a. a waist band having a front side and a back side;
b. at least one pocket flap, the pocket flap coupled to the front side of the waist band and adapted to form a pocket in combination with the waist band to secure and to support a medical drainage reservoir; and
c. a tubing aperture extending through the pocket, the tubing aperture adapted to receive a medical tubing and enable the medical tubing to deliver fluid to the medical drainage reservoir while secured by the pocket flap.

2. The belt of claim 1, wherein said pocket flap is partly fixed to the waist band and partly detachably coupled to the waist band.

3. The belt of claim 1, wherein the waist band is adapted to encompass a human waist circumference of at least 18 inches.

4. The belt of claim 1, further comprising at least one releasable fastener, the fastener adapted to at least partial decouple the pocket flap and the waist band.

5. The belt of claim 4, wherein the at least one releasable fastener is adapted to enable complete decoupling of the pocket flap and the waist band.

6. The belt of claim 1, wherein at least one tubing aperture is positioned to receive medical tubing extending through the waist band.

7. The belt of claim 6, wherein the at least one tubing aperture is adapted to enable insertion of a medical drainage reservoir.

8. The belt of claim 6, wherein said pocket flap is partly fixed to the waist band and partly detachably coupled to the waist band.

9. The belt of claim 6, wherein the waist band is adapted to encompass a human waist circumference of at least 18 inches.

10. The belt of claim 6, further comprising at least one releasable fastener, adapted to enable at least partial decoupling of the pocket flap and the waist band.

11. The belt of claim 10, wherein the at least one releasable fastener is adapted to enable complete decoupling of the pocket flap and the waist band.

12. The belt of claim 1, further comprising an examination aperture, configured to allow removal of the drainage reservoir while the medical tubing extends into the pocket.

13. The belt of claim 1, wherein the pocket flap is at least partly transparent to allow inspection of the fluid level in the drainage reservoir.

14. The belt of claim 1, comprising a pocket flap wherein elastic material forms the top of said pocket flap so that the top of the pocket flap retracts.

15. The belt of claim 1, comprising a pocket flap configured to secure and store a body fluid drainage reservoir.

16. The belt of claim 1, comprising an insertable rigid shield.

17. The belt of claim 1, wherein the pocket flap is coupled to the waist belt proximate to the wearer's torso.

18. The belt of claim 1, further comprising a plurality of pocket flaps, each pocket flap adapted to form a separate pocket in combination with the waist band.

19. The belt of claim 4, wherein the releasable fastener comprises an element selected from the group consisting of snaps, buttons, ties, hook and loop material, hook and eye material, buckles, and zippers.

20. A method, comprising:
a. providing a belt having a pocket configured for securing a fluid reservoir, the pocket comprising a tubing aperture, the tubing aperture enabling placement and removal of a fluid reservoir from within the pocket, the belt adapted for removable placement about a user's torso;
b. coupling a fluid reservoir to a wearer's body; and
c. placing the fluid reservoir into the pocket, whereby a fluid tubing extends through the tubing aperture and delivers fluid into the fluid reservoir.
